(19) United States
(12) Patent Application Publication
(10) Pub. No.: US 2006/0240081 A1
(43) Pub. Date: Oct. 26, 2006

(54) Pack-a-poof

(76) Inventor: Colleen Kelly, Export, PA (US)

Correspondence Address: JAMES RAY & ASSOCIATES, 2640 PITCAIRN ROAD, MONROEVILLE, PA, 15146, US

(21) Appl. No.: 11/113,619

(22) Filed: Apr. 25, 2005

(51) Int. Cl. A61K 9/70

(52) U.S. Cl. 424/443

(57) ABSTRACT

A disposable apparatus for body cleansing comprising a preselected material, which exhibits both flexible, softness and porous properties, formed into a predetermined shape and a cleansing material at least one of disposed on and disposed within and retained by said preselected material.

### CROSS REFERENCE TO RELATED APPLICATIONS

This application is closely related to U.S. patent application Ser. No. 11/018,461 entitled Pack-A-Poof, filed Dec. 21, 2004 and U.S. patent application Ser. No. ______, also entitled Pack-A-Pouf, filed concurrently herewith.

### FIELD OF THE INVENTION

The present invention relates, in general, to a cleansing apparatus and, more particularly, the present invention relates to a disposable cleansing apparatus made of a polymer netting which includes a capsule containing liquid or gel type soaps encapsulated therein.

### BACKGROUND OF THE INVENTION

In recent years, there has been a major change in shower products. The puff (or “poof”) has been used as a cleansing agent as a shower tool for many consumers. When used in combination with a shower gel/soap it has become an indispensable tool because of the advantages it possesses over traditional wash cloth/bar soaps. The puff provides a way of washing with less mess in the shower. It also provides gentle exfoliation and produces a greater amount of soapsuds using a lesser amount of soap product than traditional cloths/bar soaps. However, the puff does have drawbacks. First and foremost, it does not travel well. When it is packed with a bottle of shower gel, it becomes rather bulky. Further, the puff becomes saturated with water during use and, thus, it must be placed in a plastic bag after use before being packed in the suitcase. Being wet, it can also be a breeding ground for bacteria. Another alternative is to simply discard the puff; however, with presently available puffs this may not be economically feasible. Thus, there is a need for an improved puff or poof, which can eliminate the drawbacks found with present poofs.

### SUMMARY OF THE INVENTION

In a first aspect the present invention provides a disposable apparatus for body cleansing comprising a preselected material, which exhibits both flexible, softness and porous properties, formed into a predetermined shape and a cleansing material at least one of disposed on and disposed within and retained by said preselected material.

In another aspect of the invention there is provided a disposable package for body cleansing. The disposable package includes a disposable apparatus for body cleansing which has a preselected material exhibiting flexible, softness and porous properties formed into a predetermined shape and a cleansing material at least one of disposed on and disposed within and retained by the preselected material. there is also a container for containing such disposable apparatus for body cleansing.

In yet another embodiment of the invention there is provided a disposable apparatus for surgical cleansing comprising a preselected material bunched together in a predetermined shape and at least one capsule formed from a frangible containment material and having at least one of a liquid medicated soap and a medicated gel encapsulated therein which is one of disposed on and disposed within and retained by said preselected material.

### OBJECTS OF THE INVENTION

It is, therefore, one of the primary objects of the present invention to provide a disposable “poof” which contains a capsule of liquid soap or shower gel.

Another object of the present invention is to provide a disposable poof with a frangible material to form the capsule with which to enclose the liquid soap or shower gel.

It is another object of the present invention to provide a disposable poof in which the frangible material can be readily punctured upon the application of a force thereto to release the shower gel or liquid soap.

Yet, another object of the present invention is to provide a disposable poof which uses a polymer netting which is relatively inexpensive.

Still another object of the present invention is to provide a disposable poof which is convenient and travel-sized.

Another object of the present invention is to provide a disposable poof which is smaller than traditional poofs.

It is still another object of the present invention to provide a disposable cleansing apparatus which contains medicinal soap or gel for surgical cleansing.

In addition to the various objects and advantages of the invention which have been described in some specific detail above it should be noted that various other objects and advantages of the present invention will become more readily apparent to those persons who are skilled in the relevant art from the following more detailed description, particularly, when such description is taken in conjunction with the attached drawing Figures and with the appended claims.

### BRIEF DESCRIPTION OF THE DRAWINGS

FIG. 1 is a partial perspective view of the disposable apparatus according to an embodiment of the invention packed in a container.

FIG. 2 is a partial perspective view of the disposable apparatus showing the encapsulated shower gel or liquid soap.

FIG. 3 is a partial perspective view of the apparatus according to an alternate embodiment of the invention.

FIG. 4 is a partial perspective view of the disposable apparatus showing the thread used for securing the net like material.

FIG. 5 is a partial perspective view of the disposable apparatus showing an alternative means for disposing the encapsulated shower gel or liquid soap on the poof.

FIG. 6 is a partial perspective view of the disposable apparatus according to an alternative embodiment of the invention packed in a container.

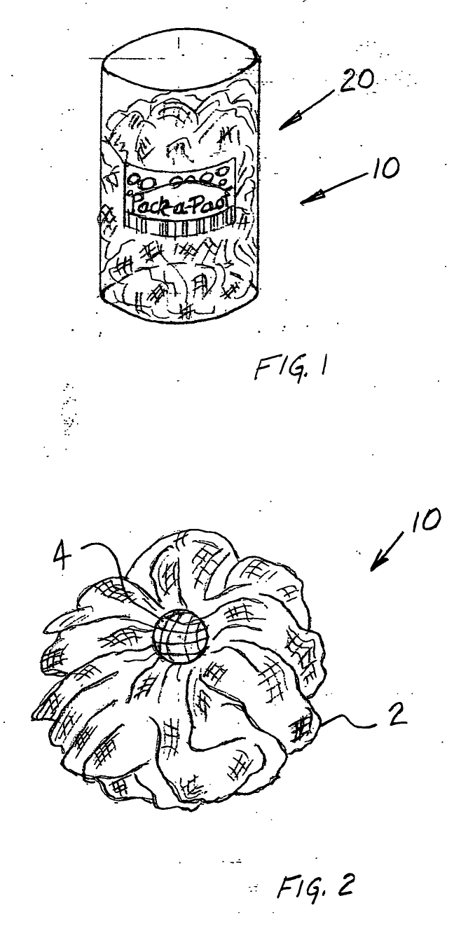

### BRIEF DESCRIPTION OF THE PRESENTLY PREFERRED AND VARIOUS ALTERNATIVE EMBODIMENTS OF THE INVENTION

Prior to proceeding to the more detailed description of the invention, it should be noted that identical components having identical functions have been designated with identical reference numerals throughout the several views illustrated in the drawings for the sake of clarity.

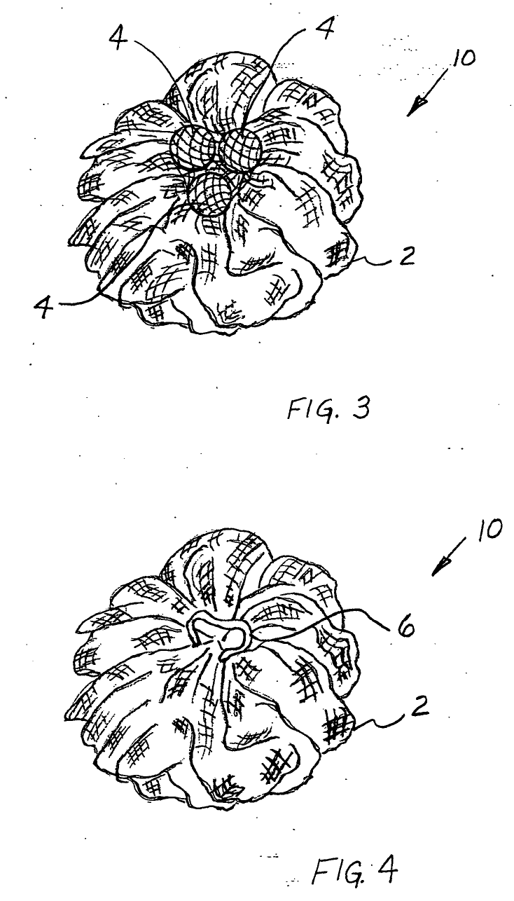

Now reference is made, more particularly, to drawing FIGS. 2 and 3. Illustrated therein is a disposable apparatus called a poof, generally designated 10, for body cleansing. Traditional poofs 10 or puffs are used to cleanse the body, and provide the benefits of gentle exfoliation and robust soapsuds. The poofs 10 provide a distinct advantage over washing with conventional bar soaps and washcloths or even with shower gels. Pouring a shower gel directly into ones hand isn't effective and sliding off the hand loses much of the gel.

The “poof” 10 of the present invention is a sponge like object constructed of a flexible material 2. It is preferred that such flexible material is a net like material and even more preferred that such net like material is a polymer netting 2. Such poof 10 contains a plurality of layers of such polymer netting 2 bunched together in a predetermined shape to form such poof 10.

There is further a means 6 engageable with such poof 10 for securing such plurality of layers of the net like material 2. It is presently preferred that such means for securing the net like material 2 be one of thread 6 as is seen in FIG. 4. Such thread 6 is tied together. It is also presently preferred that the predetermined shape of the poof 10 is substantially spherical. The poof 10 includes a capsule 4 which is formed from a frangible material and has a shower gel or a liquid soap encapsulated therein. The frangible material can be formed from any well-known material such as plastic, water-resistant treated paper, foil, and the like. The material is such that it may be readily punctured or burst upon the application of a squeezing force thereto so as to release the encapsulated cleansing material into the poof. Thus, the poof 10 contains all that is necessary for cleansing in a shower or bath. The capsule 4 retains the shower gel or liquid soap until the user is ready to use it. The capsule 4 with the gel or soap enclosed therein has a predetermined shape. In a presently preferred embodiment of the invention such shape is substantially spherical, however, it is within the scope of the invention that such predetermined shape could be virtually any shape that would be desired.

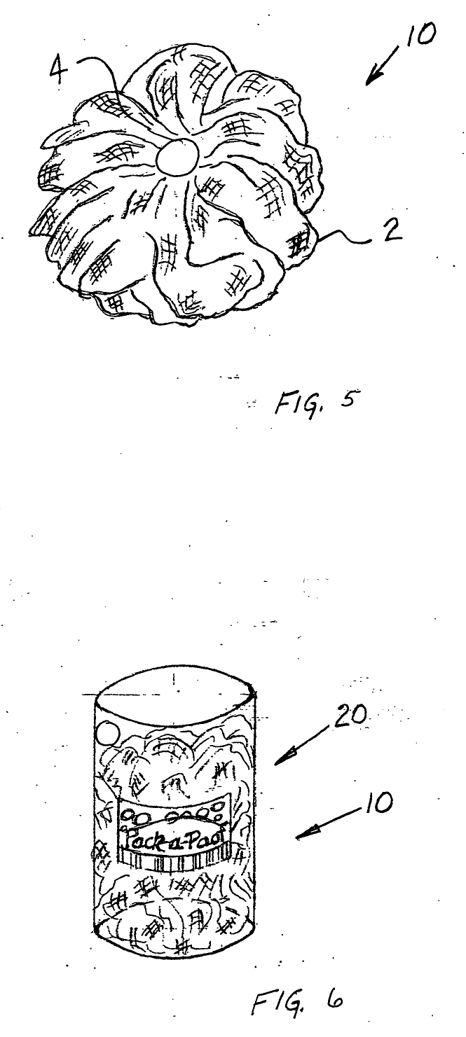

As shown in FIGS. 2 and 3, the capsule 4 can be disposed within and retained by a portion of the netting material of the poof. Alternatively, FIG. 5 shows the capsule 4 disposed on the poof. The capsule 4 can be secured to the poof by any well known means such as such as of gluing, fusing, sewing and the like.

In an alternate embodiment of the invention the apparatus includes more than one capsule 4 with the shower gel or liquid soap enclosed therein. As seen in FIG. 3 a poof 10 is shown wherein there are three capsules 4 with soap inside. In the case where more than one capsule 4 are included in the poof 10, these capsules can be punctured as different times while showering so as to release the second shower gel after the first shower gel has already been released and possibly used up. Alternatively, different types of shower gels may be contained within the different capsules e.g. a fragrance containing gel, a moisturizing gel, and the like.

In still another alternate embodiment of the invention there is provided a disposable package, generally designated 20, for containing the disposable poof 10. The disposable poof 10 would be the same as that described above. It is presently preferred that such disposable package 20 is a plastic bag, however any other well known bag material may be used such as water-resistant treated paper, foil, and the like. FIG. 6 shows yet another alternative of the invention wherein the capsule 4 is not secured to the poof 10, but rather is provided as a separate member within the package 20.

Yet in another embodiment of the invention there is provided a disposable cleansing apparatus 10 similar to the poof 10 described previously except this embodiment is designed for use in surgical scrubbing. The basic difference in this cleansing apparatus 10 is that the capsule 4 contains a medicated liquid soap or a medicated gel for such surgical cleansing. It should be noted that such medicated liquid soap and medicated gel includes anti-microbial, anti-bacterial, povidone-iodine (betadine), antiseptic, surgical soap, and sterilizing soap.

Thus, the present invention provides a disposable poof 10 with a shower gel or liquid soap encapsulated within a capsule 4 for use as a cleansing agent and then is thrown away. Such disposable poof 10 is carried in a plastic bag 20 which is also disposable. Since the poof 10 is disposable the problem with packing a wet poof is eliminated since the poof 10 is just discarded after use. Also the danger of the wet stored poof being a breeding ground for bacteria is also eliminated since there is no storage of wet poofs 10.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

I claim:

1. A disposable apparatus for body cleansing, said disposable apparatus comprising:
(a) a preselected material, which exhibits both flexible, softness and porous properties, formed into a predetermined shape; and
(b) a cleansing material at least one of disposed on and disposed within and retained by said preselected material.

2. A disposable apparatus for body cleansing as recited in claim 1, wherein said preselected material includes a plurality of layers of a polymer type material bunched together to form a poof and said disposable apparatus further includes a means engageable with said plurality of layers of polymer type material for securing said plurality of layers together and said cleansing material is selected from the group consisting of liquid soap, a shower gel and combinations thereof.

3. A disposable apparatus for body cleansing as recited in claim 2, wherein said cleansing material is encapsulated within a frangible containment material to form at least one capsule.

4. A disposable apparatus for body cleansing as recited in claim 3, wherein said at least one capsule can be readily punctured by an application of force thereto to release said cleansing material onto said poof.

5. A disposable apparatus for body cleansing as recited in claim 3, wherein said frangible containment material is selected from the group consisting of plastic, treated paper, foil material and combinations thereof.

6. A disposable apparatus for body cleansing as recited in claim 1, wherein said preselected material is a flexible netting material.

7. A disposable apparatus for body cleansing as recited in claim 6, wherein said flexible netting material is an organic polymer netting.

8. A disposable apparatus for body cleansing as recited in claim 2, wherein said means for securing said plurality of layers of said polymer type material in said form of said poof is thread.

9. A disposable apparatus for body cleansing as recited in claim 8, wherein said thread is a synthetic fabric.

10. A disposable apparatus for body cleansing as recited in claim 9, wherein said synthetic fabric is nylon.

11. A disposable apparatus for body cleansing as recited in claim 3, wherein said disposable apparatus includes a plurality of capsules disposed at least one of on and within and retained by said preselected material.

12. A disposable apparatus for body cleansing as recited in claim 3, wherein said disposable apparatus further includes a means for securing said at least one capsule to said poof.

13. A disposable apparatus for body cleansing as recited in claim 12, wherein said means for securing said at least one capsule to said poof is one of gluing, fusing and sewing.

14. A disposable apparatus for body cleansing as recited in claim 3, wherein said at least one capsule is enclosed and retained within a portion of said preselected material.

15. A disposable package for body cleansing, said disposable package comprising:
(a) a disposable apparatus for body cleansing, said disposable apparatus including:
(i) a preselected material, which exhibits each of flexible, softness and porous properties, formed into a predetermined shape; and
(ii) a cleansing material at least one of disposed on and disposed within and retained by said preselected material; and
(b) a container for containing said disposable apparatus for body cleansing.

16. A disposable package for body cleansing as recited in claim 15, wherein said preselected material is a plurality of polymer type materials and said disposable package further includes a means engageable with said plurality of layers of polymer type material for securing said plurality of layers together to form a poof and said cleansing material is contained within a capsule which is one of disposed on and disposed within and retained by said polymer type material.

17. A disposable package for body cleansing as recited in claim 16, wherein said capsule is placed within said container for containing said disposable apparatus for body cleansing.

18. A disposable package for body cleansing as recited in claim 16, wherein said at least one capsule can be readily punctured by an application of force thereto to release said cleansing material onto said poof.

19. A disposable package for body cleansing as recited in claim 18, wherein capsule is a frangible material selected from the group consisting of plastic, treated paper, a foil material and combinations thereof.

20. A disposable package for body cleansing as recited in claim 15, wherein said container is one of plastic, treated paper and a foil bag.

21. A disposable apparatus for surgical cleansing, said disposable apparatus comprising:
(a) a preselected material bunched together in a predetermined shape; and
(b) at least one capsule formed from a frangible containment material and having at least one of a liquid medicated soap and a medicated gel encapsulated therein which is one of disposed on and disposed within and retained by said preselected material.

22. A disposable apparatus for surgical cleansing as recited in claim 21, wherein said at least one of a liquid medicated soap and a medicated gel are selected from the group consisting of anti-microbial, anti-bacterial, povidone-iodine, antiseptic, surgical soap, and sterilizing soap.

23. A disposable apparatus for surgical cleansing as recited in claim 21, wherein said at least one capsule can be readily punctured by the application of a force thereto to release at least one of said liquid medicated soap and medicated gel onto said poof.
